(19) United States
(12) Patent Application Publication
(10) Pub. No.: US 2010/0016663 A1
(43) Pub. Date: Jan. 21, 2010

(54) Device for the Guidance of Instruments in Cavities

(76) Inventors: Bernhard Maisch, Marburg (DE); Heinz Rupp, Marburg (DE); Thomas Philipp Rupp, Marburg (DE)

Correspondence Address: CLARK & BRODY, 1090 VERMONT AVENUE, NW, SUITE 250, WASHINGTON, DC, 20005, US

(21) Appl. No.: 12/085,464

(22) PCT Filed: Nov. 30, 2006
(86) PCT No.: PCT/DE2006/002116

§ 371 Date: Sep. 23, 2009

(30) Foreign Application Priority Data
Nov. 30, 2005 (DE) 10 2005 057 479.3

(51) Int. Cl. A61B 1/01

(52) U.S. Cl. 600/114

(57) ABSTRACT

The invention relates to an arrangement which can be fixed to the inside of a cavity by means of fixing means which are associated with said arrangement. Said fixing means fix the arrangement in relation to at least one part of the wall of the cavity which is to be examined, wherein the arrangement must at least partially be inserted. Said arrangement enables the instruments, which are used to examine/manipulate cavities, even when the arrangement and/or the instrument is displaced in a translatory and/or rotational manner, in particular, in angled cavities, to be guided in a reliable manner.

### DESCRIPTION AND STATE OF THE ART

The invention concerns a device for the guidance of instruments, in particular medical instruments, such as e.g. endoscopes, extraction or mounting tools or tools for the optical visualization of selected areas, in cavities. Cavities are understood to be those in animals, as well as in technical devices, such as e.g. cavities in turbines.

The device serves, in particular, for the guidance of flexible instruments, so that, through bending of the instruments or the device, the instruments can also be guided around corners.

For the guidance and also the bending of instruments, in particular medical instruments, numerous devices are already known.

For example, the endoscope described in DE 197 27 419 belongs to this category. Flexible endoscopes or those that can be formed or guided around a corner have likewise already been known for a while. An example of this is the endoscope described in DE 200 03 207.

An “endoscopic intubation system” belonging to this group is also known from DE 201 05 206.

In a more technical area, the example from DD 282 829 of a device for the guidance of flexible endoscopes should be named.

In the aforementioned examples, the bending of the instruments is realized primarily by cored wires.

### DISADVANTAGES IN THE STATE OF THE ART

All of the previously known devices comprise the disadvantage that guidance of instruments with the known devices can indeed be very exact within the device, however guidance relative to the walls of the cavities is, in most cases, only unreliably possible. Only in the cases where the external diameter of the device, in which the instruments are guided, coincidentally corresponds to the internal diameter of the cavities, is exact guidance of the instruments relative to the walls of the cavity possible as well. Often then, an optimal position found once, after movement of the devices or instruments as well is not found again, or uncontrolled movements of the devices or the instruments guided within occur. If the instruments serve, as is mostly the case, for the execution of punctures or the insertion of probes or sensors, these movements can easily lead to false positioning or to injuries/damage of the walls of the cavities.

Another disadvantage of the known devices is that the feeding of the devices occurs mostly in an uncontrolled manner, e.g. in predominantly canal-shaped cavities except for the coincidental guidance through the walls of the cavities.

### AIM OF THE INVENTION

The aim of the invention at hand is then the provision of a device which enables the prerequisites for reliable guidance of instruments relative to at least one part of the walls of a cavity.

In the case of medical or veterinary instruments, only by such a device is e.g. the defined examination or/and reliable puncture or/and other manipulation or/and visualization of human or animal vessels or tissues possible.

The given aim is achieved, according to the present invention, by a device according to claim 1.

In order to solve the existing problems, a device according to the present invention was developed which allows fixation of the device within the cavity via attachment means assigned to the device. The attachment means provide for fixation of the device relative to at least one part of the wall of the cavity to be examined, in which the device has to be at least partially inserted.

Hereby, the attachment means are realized in such a way that they come into contact with at least one part of the cavity wall upon activation, e.g. frictional connection, and thus fix the device relative to this part of the wall.

The device fixed in this way offers improved attachment for subsequent movement of the instrument, which is, e.g., guided within the device in order to conduct reliable manipulations or visual examinations etc. within the cavity or starting from its walls.

For improvement of the guidance in forwards or backwards movements of the instruments a first embodiment of the device according to the present invention likewise offers much improved guidance compared to the state of the art. For this, the device comprises at least two attachment means spaced from each other in the direction of the desired movement (e.g. forwards or backwards) which are attached to the device and realized in such a way that they can be activated one after another for the guidance of the forward movement.

Furthermore, the device comprises therefore means of forward movement. These are realized in such a way that after activation of the first attachment means and activation of the means of forward movement, a part of the device which is not attached is moved in the direction of the desired movement.

Attachment means are understood to also include glues or other chemical substances which dissolve after a certain time or under the influence of chemical or physical influences, such as with solvents or electromagnetic radiation or variations in temperature. These are released from the surface of the device in a sufficient amount and form an attachment after a certain length of time, which is subsequently degraded actively by the aforementioned chemical or physical influences or passively by waiting.

The means of attachment can include mechanical or electrical positioning elements, such as levers or piezoelectric or other actuators. It is advantageous in this to keep in mind that the corresponding gears or means of attachment themselves provide feedback about the pressure exerted on the walls of the cavities or the degree of attachment, in order to avoid damage or injuries. Advantageously, this feedback occurs in the case of the gears via the observation of the consumption of energy or power or impulse or tension/electricity in the course of time.

This has the advantage that no further sensors are necessary to detect the pressure in situ, namely in the cavity directly.

The device according to the present invention is suitable for being used for every human or animal tissue or organ, above all in the realm of minimally invasive surgery where there is no direct visual control and it has to be ensured that a tissue or organ is attached to a special device, e.g. to a puncture device, for a manipulation.

The device according to the present invention is naturally also suitable for being used itself as an instrument for the execution of manipulations to the walls of a cavity or the areas located behind them.

### EMBODIMENTS

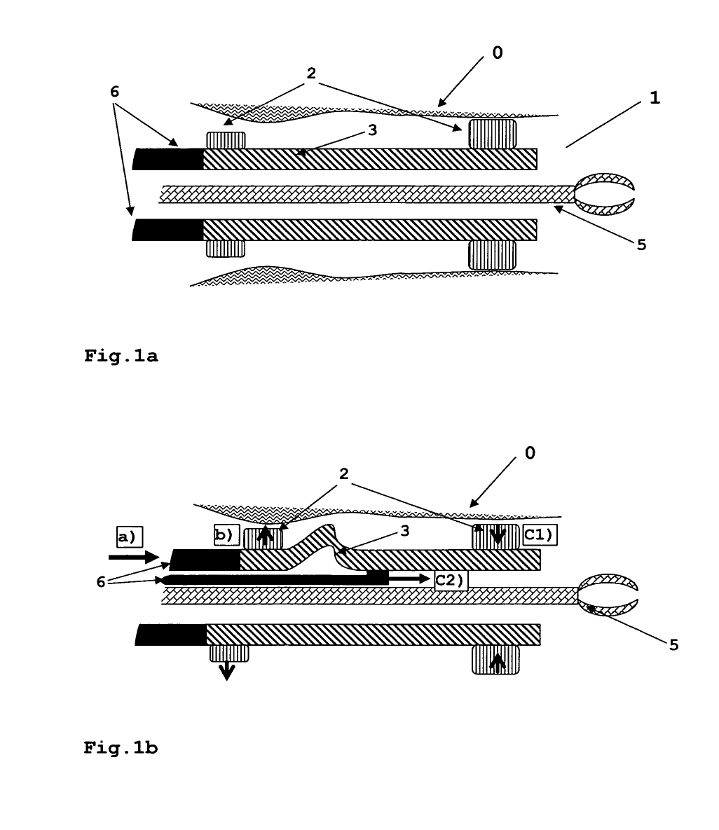

Embodiments are depicted in the FIGS. 1 to 13:

FIG. 1a shows a cross-section of the device according to the present invention in a first embodiment.

FIG. 1b shows the device from FIG. 1a, likewise in cross-section, during a movement to the right.

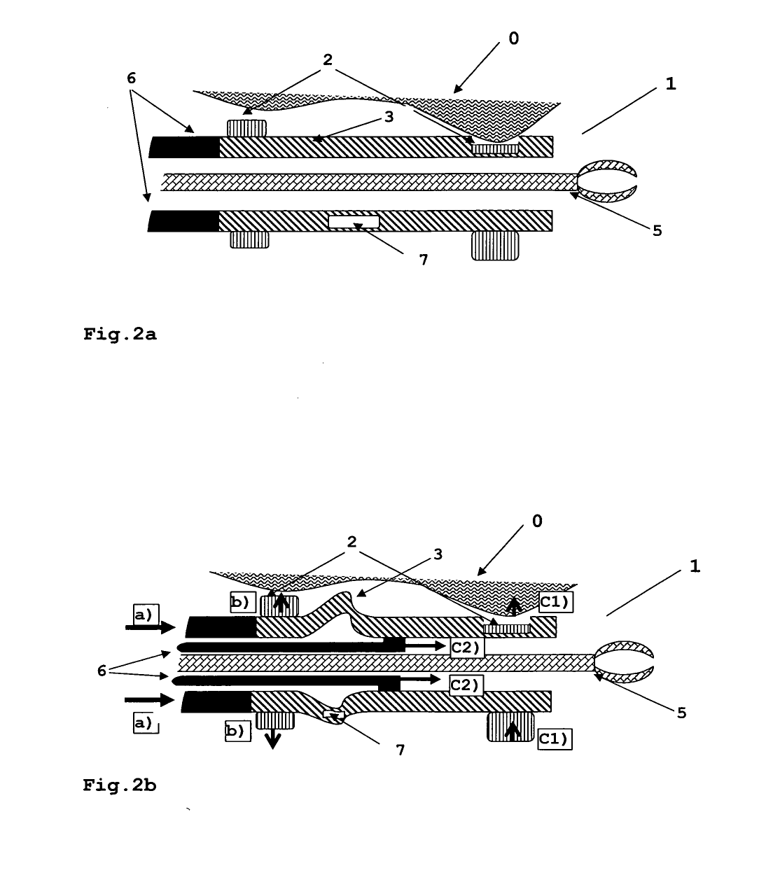

FIG. 2a shows a cross-sectional view of a further advantageous embodiment.

FIG. 2b shows the device from FIG. 2a, likewise in cross-section, during a movement to the right.

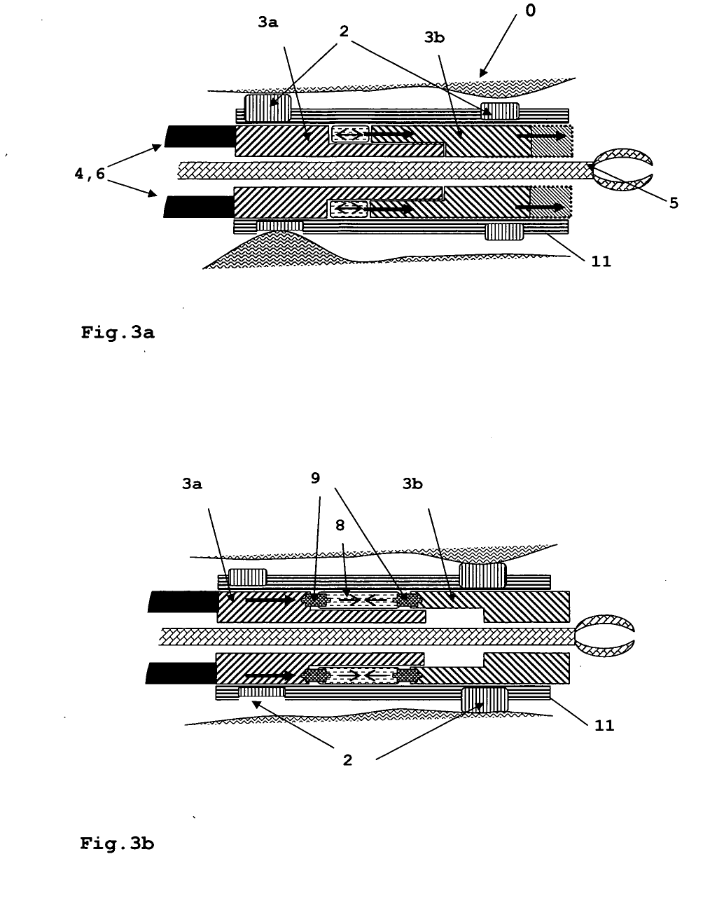

FIG. 3a shows a sectional view of a further particularly advantageous embodiment.

FIG. 3b shows the device from FIG. 3a during a movement to the right.

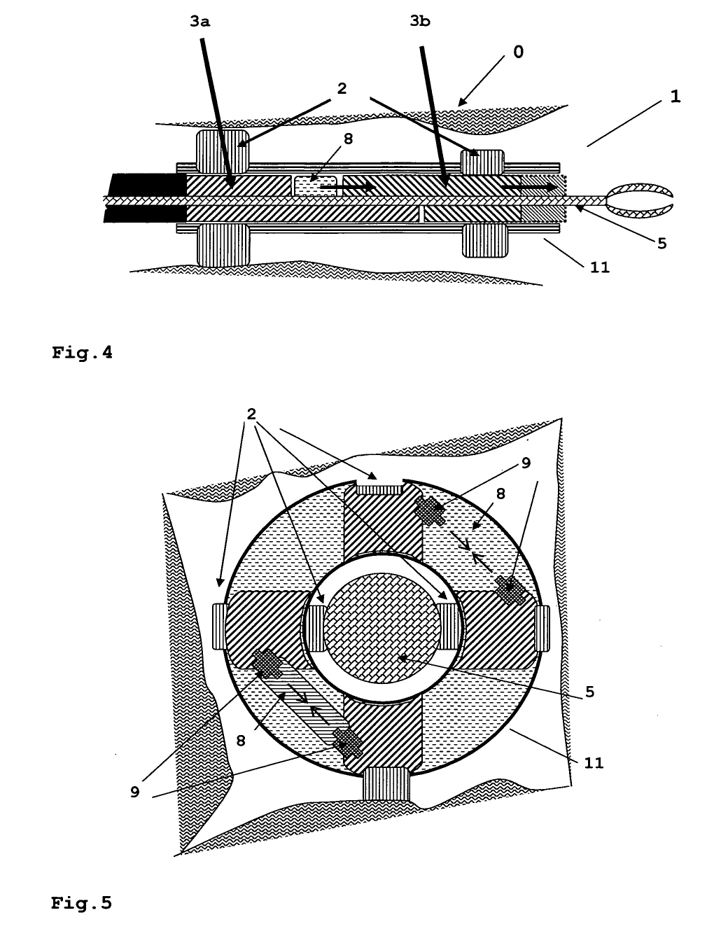

FIG. 4 shows a sectional view of a particularly slender embodiment.

FIG. 5 shows a sectional view of a rotationally symmetrical embodiment of the device according to the present invention.

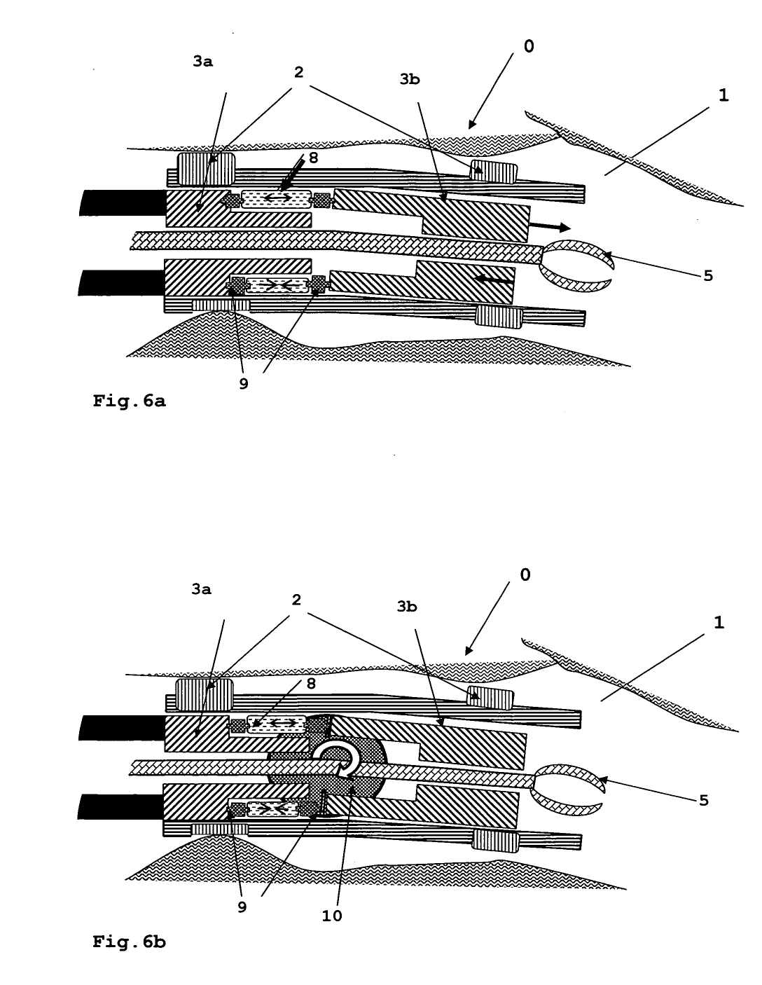

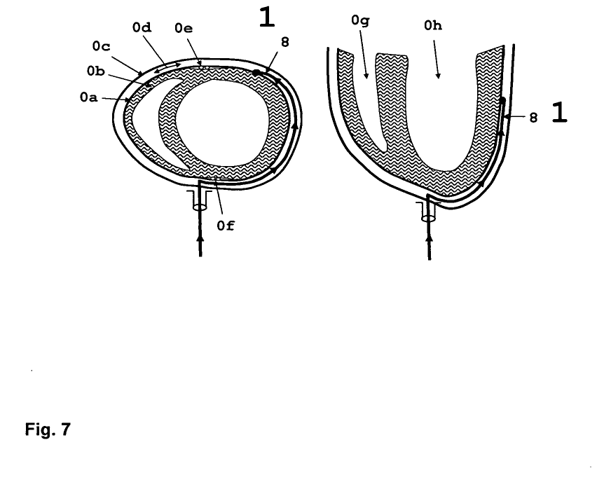

FIGS. 6a and b show a sectional view of an even more particularly advantageous embodiment of the device according to the present invention.

FIG. 7-13 show drawings for an embodiment which allows controlled guidance of instruments, preferably in the heart sac of the human or animal body.

The figures are described in more detail in the following:

FIG. 1 shows the walls (0) of a cavity, as well as the device (1) inserted therein, which guides an instrument (5). The instrument is suitable for being attached to the device by frictionally connection or repeatedly attachable and/or dissolvable. In this elongated embodiment pictured, the device comprises outer walls (3) which guide the instrument, at least in part, directly or indirectly by spacing elements which are not illustrated. Attachment means (2) are assigned to the walls (3). Assigned outer means of forward movement (6) are depicted on the wall (3) to the back, i.e. to the left in FIG. 1a. These can be realized, e.g., by rigid, single rods or a rigid wall. The means of forward movement are also suitable for being made from other materials, as long as these provide for an at least partial movement of the device or a part of the device by a push from right to left or vice versa due to the push applied outside of the device.

In FIG. 1a, the attachment means are activated in the fore, right area of the device in the form of an expansion over the surface of the wall (3). The expansion or extension over the surface of the wall is realized in such a way that clamping of the device in the fore part with the cavity wall occurs. The corresponding supply cables or recesses or canals for running the attachment means, for instance in the form of electric cables or recesses for electric gears or canals for the supply or removal of gases or fluids under pressure are illustrated neither here nor in the following figures.

FIG. 1b shows the situation, starting from that in FIG. 1a, after application of a push on the upper means of forward movement (6). The material of the wall (3) of the device is realized to be so elastic or malleable that it leads to deformation when a certain force of push is exceeded and as a result of this shortening or compression of the wall (3) of the device occurs.

After this shortening, as shown in FIG. 1b by the letters, subsequently to b) both left attachment means (2) are then activated in such a form that clamping of the device in the corresponding area of the cavity results; subsequently c1) both right attachment means are activated in such a form that the clamping of the device depicted in FIG. 1a in the right area is lifted. According to the respective composition of the wall (3) material, stretching and, therefore, elongation of the upper wall to the length value before exertion of the push subsequently occurs.

If the material of the wall is solely malleable, then the stretching in step c2) is effectuated by operation of the means of forward movement (6) which is depicted in FIG. 1b adjacent to the instrument.

Relocation of the wall towards the right results from step c1) or/and c2) and, thus, a forward movement of the device with simultaneous guidance of the instrument (5).

If no shortening and stretching in the lower region of the wall is caused, subsequent to the steps c1) or/and c2), tipping can be observed.

This tipping within the cavity can be likewise caused or increased by activation of the attachment means in differing strengths in the sense of an expansion over the surface of the wall.

In the case of an elastic or malleable wall (3), bending the device is also thus achievable.

FIG. 2 shows an advantageous embodiment of the device according to the present invention with walls constructed to be adjustable in at least their length or also in their width (not illustrated). In this, as depicted in FIGS. 2a and 2b, means for the creation of a vacuum are used as attachment means (2) in the right area of the device. Suction cups or, generally, all means which create attachment to at least one part of the cavity wall through application of a vacuum or reduction of pressure can be considered as means of creating pressure.

FIG. 2a shows how attachment of the device in the right (fore) area was created by suction of a part of the wall. Furthermore, an element for volume reduction (7) is depicted in FIG. 2a within the lower wall of the device, whereby this wall is realized to be variable in its length.

In FIG. 2b, the same procedure is shown for the upper wall as in FIG. 1b, however in this embodiment, the attachment occurred through a vacuum creating attachment means. The shortening of the wall is again clearly recognizable, i.e. the compression of the wall which is then lifted after execution of steps b) and c1) or c2).

The lower part of the wall (3) does not undergo such a large deflection as the upper part, as the element for volume reduction (7), which, e.g., is suitable for being realized as an elastic cell which is able to be reduced in volume through creation of a vacuum or as a piezoelement, provides for shortening of the lower part of the wall (3).

Relocation of the device to the right occurs here as well—as in FIGS. 1a and 1b—through all variants c1 or/and c2.

FIG. 3 shows a further advantageous embodiment of the device according to the present invention in which the wall is realized between the at least two attachment means, which are arranged spaced from each other, in at least two parts (3a, 3b) which are able to be slid against one another.

FIG. 3a shows the device after operation of both the left attachment means in such a form that attachment of the device results in the left area. After this attachment, activation of the inner means of forward movement occurs in such a form that these expand in such a way that the right parts (3b) of the wall (3) are pushed to the right (depicted through bold arrows).

FIG. 3b then shows the device after detachment of both left attachment means and after activation of attachment in the right area of the device.

For forward movement of the device (1) to the right, under guidance of the instrument (5) to the right, inner means of forward movement (8) are activated—as depicted in FIG. 3b through the bold arrows—which pull the left part (3a) of the wall (3) to the right towards the attached right area of the wall.

The inner means of forward movement (8) can be realized for this in the form of elastic elements, of elements which are suitable for being reduced in volume or/and elongation, amongst other things, parallel to the instrument, i.e. fundamentally in one direction in space, such as tubes or in the form of piezoelements etc.

In the case where only one expansion or contraction is possible, the inner means of forward movement (8) is assigned a spring or another element for prestressing (9). In FIG. 3b, the force action from the inner means of forward movement (8) is transmitted to the parts (3a, b) through corresponding elements for transmission of force (9). These are not, however, absolutely necessary, as long as another attachment between the means (8) and the parts (3a, b) can transmit the typically occurring force.

FIG. 4 shows a particularly slender, advantageous embodiment of the device (1). In this, the instrument is arranged between both parts (3a, b) of the device which are movable in opposing directions. In this embodiment, the provision of one inner means of forward movement (8) suffices. As in FIGS. 3a, 3b is also provided with an additional shell (11) outside of the walls (3a, b), which adjustably covers at least partial areas of the at least two parts of the wall which are movable in opposing directions, relative to the parts, in the corresponding embodiment of FIG. 3. Through this, fewer injuries to the cavity wall occur.

FIG. 5 shows a particularly advantageous embodiment for the circulating forward movement along the circumference. The different components of the device according to the present invention (1) are designated with the same numbers or letters as in the previous figures.

Distributed over the circumference of the wall of the device (1), the device comprises attachment means (2) which are again spaced from each other in the direction of the forward movement. As depicted below in FIG. 5, the lower means is activated and, therefore, attached to the cavity wall. Through activation of the inner means of forward movement (8) (in the bottom left quadrant of the fig.), a contraction (not illustrated) can then be caused. Through activation of the left attachment means (2) and subsequent detachment of the lower attachment means a counter-clockwise circumferential movement of the device can then be caused according to the same principles as in the aforementioned figures.

In the case that the instrument (5, in the middle of FIG. 5, depicted in cross-section) has to be correspondingly rotated together with the device, at least one further attachment means (2) is provided between the walls of the device and the instrument, which attaches the instrument to the corresponding part of the wall and rotates together with the circulating forward movement along the circumference of the device.

FIG. 6a, b show a further even more particularly advantageous embodiment of the device. In this, the device is active, i.e. able to be bended by the inner means of forward movement, in order to reliably guarantee guidance of an instrument and forward movement of the device even in bent cavities.

FIG. 6a shows a variant of this embodiment, in which the inner means of forward movement (8) are realized in a manner separated from one another and, also, without any relation between their sizes offering an expansion or reduction in at least one direction in space. The shell (11) is realized in an elastic or malleable manner for this embodiment. In order not to have to provide for restoring forces through the inner means of forward movement which are principally suitable for that, it is provided, in the corresponding cases, that the shell is chosen from a material which comprises a restoring force.

FIG. 6b shows a second variant of this embodiment in which the device (1) additionally features bending means (10) in the form of a rotatable disc. Naturally, all possible variants of joints or gears which cause bending of the at least two parts of the wall (3a, b) are suitable for being used for this.

A further variant of this embodiment in which the inner means of forward movement (8) are no longer provided, as the bending means is realized to be active, i.e. provided with its own drive, is not illustrated.

In particular with the last embodiment, it is clearly more easily possible to reliably guide an instrument also around a corner and reliably re-access a position which was accessed once, as the guidance of the instrument only allows translative or rotative movements of the instrument.

A further advantageous—not illustrated—embodiment provides for the use of sensors for the surveillance of the attachment, for instance in the form of means of pressure-surveillance or in the case of electrical attachment means, power/tension and/or energy/power surveillance means, as well as the corresponding display devices which are either visual, tactile, or stimulating another human sense.

After calibration of the device, for instance for chosen cavities in human bodies, as well as the corresponding activator and surveillance means of the attachment, it is possible to move the device with the instrument automatically to a certain position, for instance in the body of a human—after insertion into the respective cavity. This is, in particular, made possible in bent cavities by the embodiments according to FIGS. 6a and 6b and, in particular, in the combination of this embodiment with that according to FIG. 5.

For that purpose, corresponding storage devices and control/regulation signalers, for instance in the form of a PC, are to be assigned to the device.

It is immediately obvious to persons skilled in the art that the device according to the present invention (1) is also bendable by use of a bendable instrument, at least provided that the walls of the devices are realized to be flexible.

A further embodiment provides that the device is realized in such a way that a part of the attachment means is arranged on the instrument to be guided or also on a further inner wall (4) of the device which has not been separately illustrated in the previously described figures. In the case of the provision of attachment means on the inner wall (4) of the device, this is realized to be relatively movable in regard to the outer wall (3a, 3b) and comprises either inner means of forward movement (8), such as depicted e.g. in FIGS. 1, 3a and 3b or is coupled with outer means of forward movement (6), such as e.g. a bar or a wire which is compression- and stretch-resistant.

This embodiment is particularly preferred to be used for the controlled movement of instruments, preferably in the heart sac of a human or animal body. With this, the guidance of the instrument preferably occurs via a reliably recognized and signaled attachment of the outer wall (3a, 3b) or the inner wall (4) which protrudes for this purpose from the inner wall to a surrounding tissue. This embodiment also allows, for example, the attachment of stimulation electrodes at a defined location, which lies bent to the point of the instrument which is being inserted, on the surface of the heart. Available instruments are not suitable for this application, as shown by the example of a controllable endoscope. After bending the endoscope head, the following movement in the direction of the bending requires a wall which is stationary and repellent to the endoscope head. If this wall is not available, the movement succeeds only in elongation of the point from which the endoscope is pushed forward, despite the endoscope head being bent. The target destination which lies after an angle can not therefore be reached. Since in the example of the implantation of an electrode on the surface of the heart, a required wall rejecting the endoscope is not available, the guidance misses and the location suitable for the attachment of the electrode is not reached. The desired location can also not be reached solely with bending the head section of an endoscope, as the head section is too short. A longer head section would not allow bending due to the locational constrictions and injuries could not be ruled out.

In order to solve the existing problems, in this embodiment the arrangement is chosen in such a way that one part of the attachment means (2, 2a) is arranged on the outer wall (3a, 3b) of the device and one part of the attachment means to an inner wall (4) which is movable against the outer wall (3a, 3b) or on the instrument to be guided (5) itself. The two walls or the outer wall and the instrument are suitable for being moved forward either through the use of outer means of forward movement (6) (e.g. a compression-resistant and/or tightly connected wire or tube) or in combination with inner means of forward movement (8) in a temporally and spatially staggered manner. A bending necessary for the case given above of the entrance into heart vessels occurs after attachment of the outer wall (3a, b) on the surface of the heart or on the pericardium. This embodiment is suitable for being used for every human or animal tissue or organ, especially in the realm of minimally invasive surgery, without any direct visual control and where a defined location can not be reached by a conventional controllable endoscope for the reasons mentioned above.

In a modification of this embodiment, it is provided that the outer wall (3a, 3b) is realized in two sections, wherein the resulting head and main section is realized to be bound with a bending means. The bending of the head section occurs, e.g., via a control wire. The attachment of the main section necessary for the targeted bending of the head section is achieved, e.g., via mechanical immobilization or through creation of a vacuum. A detection device for attachment assigned to the device is suitable for comprising one or more means of detection, amongst others pressure-dependent, acoustic, as well as optical. By means of a display device, signals which are detected from the detection device for attachment are transformed into display signals.

Embodiments and illustrations concerning the anatomy of the heart are depicted in FIGS. 7 to 12.

FIG. 7 shows the heart in a cross-section (left) and a longitudinal section (right).

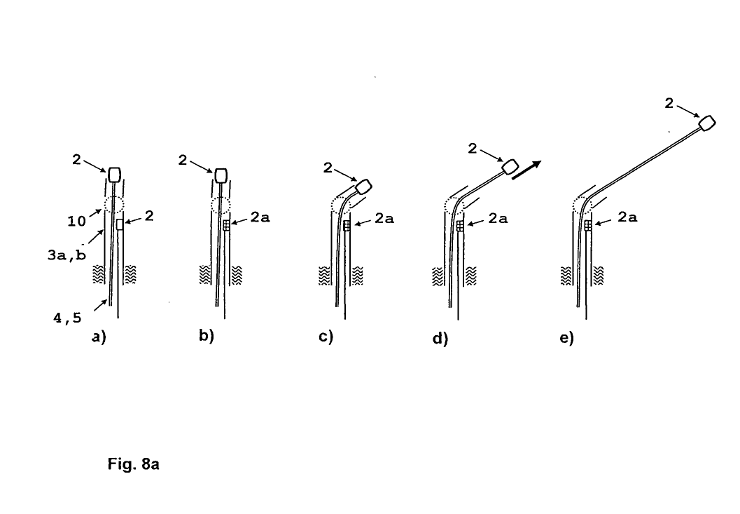

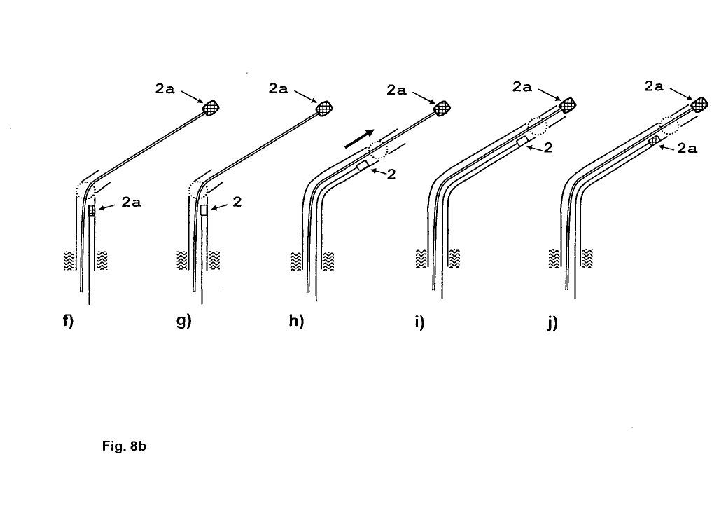

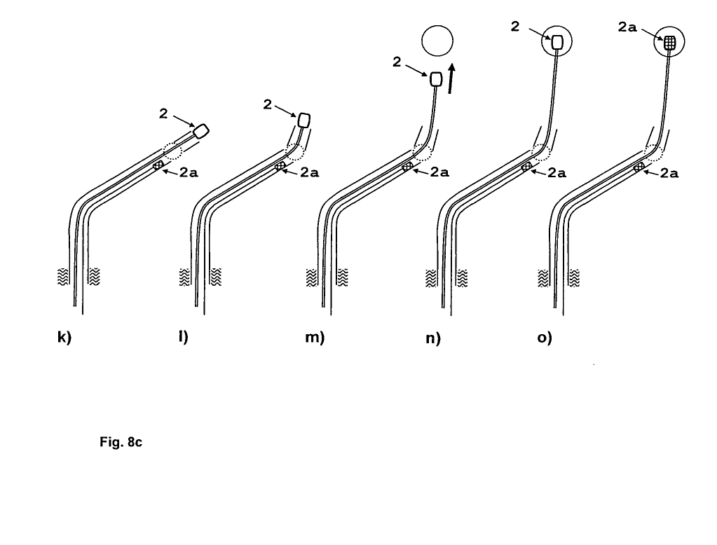

FIG. 8 schematically shows steps of the temporally and spatially staggered movements of the outer wall (3a, b) and the inner wall (4) or the instrument (5) on which a part of the attachment means (2, 2a) is arranged.

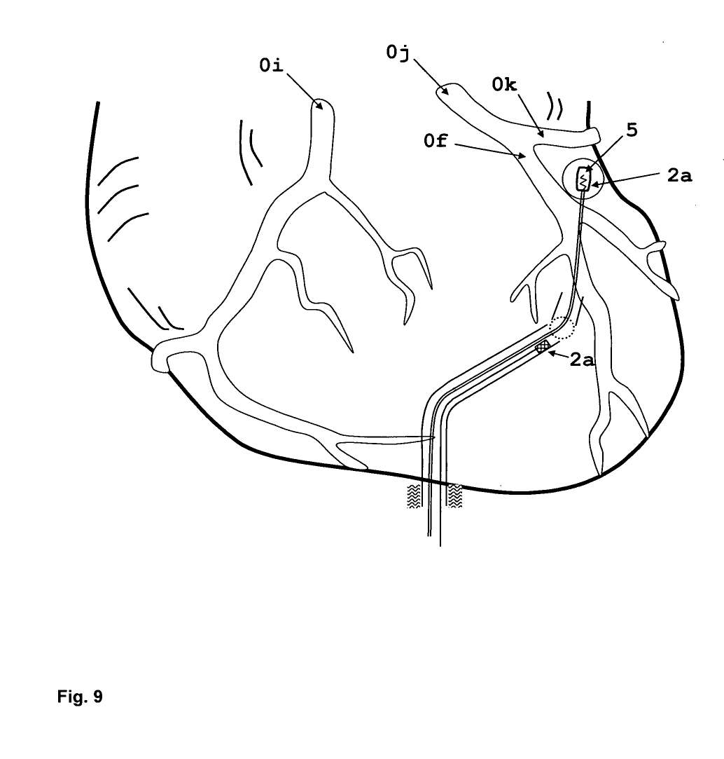

FIG. 9 shows the situation, which is depicted on the right of FIG. 8c, projected onto the surface of the heart.

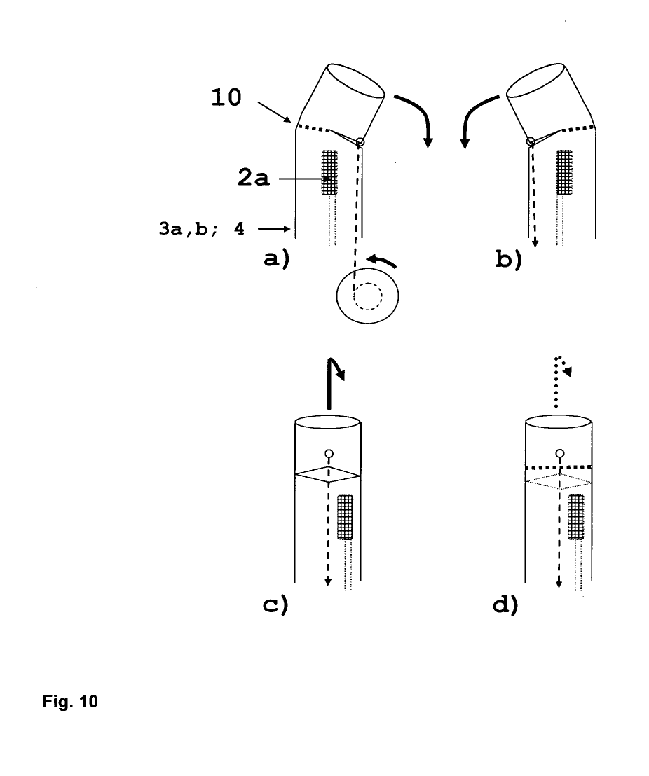

FIG. 10 shows several head sections of the outer wall (3a,b) which are movable via bending means, and which are suitable for comprising, in the main section, and, also—not illustrated—in the head section, respectively, an attachment means.

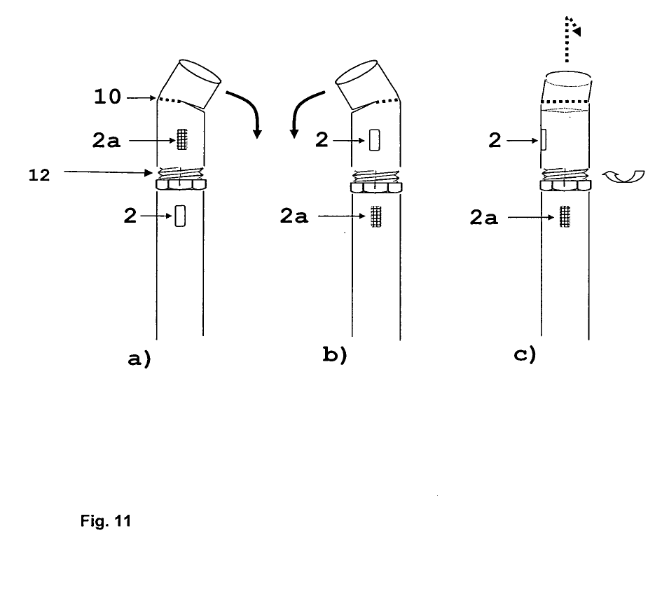

FIG. 11 shows an embodiment of the device according to the present invention, wherein the outer wall (3a, b) is realized in three sections and in the lower two sections, which are separated from each other through rotation means (here in the form of a thread), respectively comprise one attachment means. The connection between the upper two sections is realized by a bending means (10). In this particularly advantageous embodiment, the head section is thus realized to be rotatable in relation to the main section.

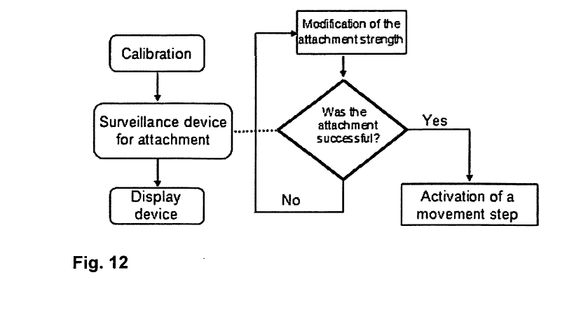

FIG. 12 shows the process and controlling means during surveillance of an activated attachment means.

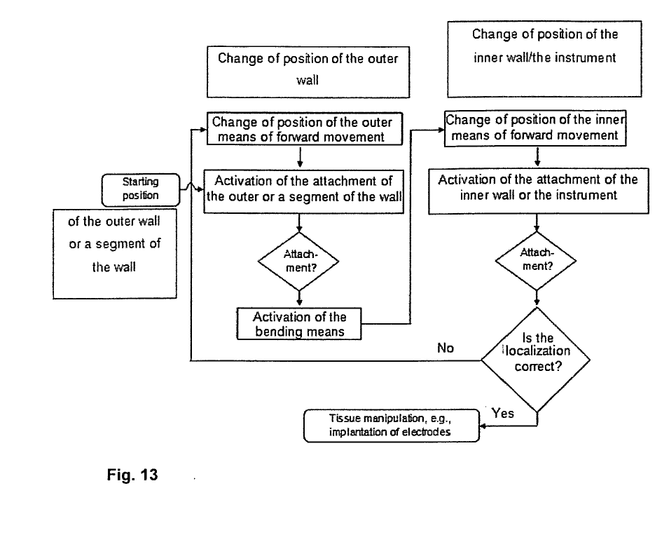

FIG. 13 shows, for instance, a process and controlling means during the movement of the outer wall and the inner wall or the instrument.

The figures are described in more detail in the following:

FIG. 7 The epicardium (0a), which lies on the surface of the myocardium (0b) and the parietal pericardium (0c) and the cavity of the heart sac (0d) are depicted. The heart coronary artery ramus interventricularis posterior (0e) which emanates from the arteria coronaria dextra and the heart coronary artery ramus interventricularis anterior (0f) which emanates from the arteria coronaria sinistra are depicted. The heart is composed of the right (0g) and the left (0h) heart chamber. Through an access (below) an instrument which is located within the device is guided to a predetermined target location.

FIG. 8a-c schematically show steps of the temporally and spatially staggered movement of the outer (3a, 3b) and the inner wall (4) or the instrument (5), preferably in the heart sac. In the partial Fig. a) of FIG. 8a, the device is being inserted into the heart sac. Both attachment means (2) are not activated. In partial Fig. b), activation of the attachment means (2a) on the outer wall (3a, b) is executed. In c), bending to the right occurs by continuous attachment (2a) with the help of a bending means. In the embodiment of a flexible tube, the head section can e.g. comprise a notch of different sizes, depending on the desired deflection angle. The head section is bent, e.g. through a control wire lying in the device. In d) and e), the movement of the inner wall or the guided instrument in the direction of the bending occurs with continuous attachment (2a). In f), the inner wall or the guided instrument is also attached (2a). In g), the attachment (2) of the outer wall (3a, b) is detached. In h) and i), the outer wall (3a, b) is moved along the attached inner wall or the attached instrument. In j), attachment (2a) of the outer wall occurs, then, in k), the attachment (2) of the inner wall or the instrument is detached. In 1), the head section of the outer wall is bent to the left, and in m) and n), the inner wall or the instrument is pushed in the direction of the bending. In o), the target destination is reached and the inner wall (4) or the instrument (5) can be attached. These steps can be repeated as desired, whereby the direction of the bending is suitable for being modified.

FIG. 9 shows the situation of the device depicted on the right in FIG. 8c projected onto the surface of the heart. The arteria coronaria dextra (0i), arteria coronaria sinistra (0j), ramus circumflexus (0k), and ramus interventricularis anterior (0f) are depicted. Tissue manipulation, e.g. an epicardial electrode implantation which is indicated by the corkscrew-like arrangement of the instrument (5), occurs with the instrument (5) pushed forward in the outer wall (3a, b) or in the inner wall (4). The wall (3a, b or 4) situated nearest to the instrument is attached (2a, in order to make the electrode head recognizable, the hatching of the activated attachment means was removed) during the implantation.

FIG. 10 shows four different head sections of outer or inner walls (3a, b or 4) which are movable by bending means (10), which possess, respectively one attachment means. Bending occurs subsequent to attachment to a wall through an attachment means. In the embodiment shown, it concerns a preferably flexible tube, which comprises a notch of different sizes depending on the desired deflection angle. The head section is bent, e.g., through a control wire lying in the device. In example a), the joint can be bent to the right, in b) to the left, in c) to the front and in d) to the back. The attachment means (2a), respectively, is located on the lower surface and builds in the embodiment an attachment on the surface of the heart. In this embodiment, four devices are used for the four desired bends.

FIG. 11 shows a further embodiment, wherein the outer or inner wall (3a, b or 4) is realized with two attachment means and one means of bending, wherein the front device is rotatable regarding the rear device. A possible inclining movement to the right is depicted a), a possible inclining movement to the left after rotation of the entire device of 180° is depicted b), and a possible inclining movement to the back after clockwise rotation of the head section in the thread of 90°, wherein during rotation, the rear device is attached c). The attachment (2a) is activated at the rear in a), b) and c), respectively. The device shown as an example allows three bendings, wherein only 2 attachment means and one thread are required. Instead of the thread, other compounds, which are rotatable against one another and simultaneously or independently on the rotation expandable, are suitable for being used as means of rotation or/and expansion (12).

FIG. 12 shows, for example, the process and controlling means during surveillance of an activated attachment means. With the help of a surveillance device for attachment and a display device, the attaching, which allows a defined movement of the outer or the inner wall (3a, b or 4) or the instrument, is controlled.

FIG. 13 An example depiction of the process and controlling means during movement of the outer or/and inner wall or the instrument which is at least partially surrounded by these. If the desired localization on, e.g., the surface of the heart is not reached, the movement is continued. If the localization is correct, tissue manipulation, e.g., occurs. The term “attachment” is explained in FIG. 12.

### List of Reference Numerals

0 wall of a cavity

0a epicardium

0b myocardium

0c pericardium

0d heart sac

0e ramus interventricularis posterior

0f ramus interventricularis posterior

0g right chamber of the heart

0h left chamber of the heart

1 device for the guidance of instruments

2,2a attachment means

3,3a, 3b outer wall of the device

4 inner wall of the device

5 instrument

6 outer means of forward movement

7 elements for volume reduction

8 inner means of forward movement

9 prestressing or force-transmitting elements

10 bending means

11 shell

12 means of rotation or/and extension

1. Device for the guidance of an instrument within a cavity (0), comprising a wall (3) for at least partial guidance of the instrument (5) through a section of the wall, wherein the device comprises attachment means (2, 2a) spaced from each other.

2. Device according to claim 1, wherein the attachment means (2, 2a) spaced from each other are realized in such a way that they are suitable for being activated separated from one another in the manner, where every attachment means (2, 2a) is realized to be independent from the other for the attachment or/and detachment of the corresponding part of the device to and/or from the wall of the cavity (0).

3. Device according to claim 1, wherein the device is realized in such a way that the device comprises at least one inner wall (4), which is movable and/or rotatable against the outer wall.

4. Device according to claim 1, wherein the device is realized in such a way that a part of the attachment means (2, 2a) is arranged on the instrument to be guided.

5. Device according to claim 3, wherein a part of the attachment means (2, 2a) which are spaced from each other is arranged on the wall (3) or/and the instrument (5) to be guided or/and the at least one inner wall (4).

6. Device according to claim 1, wherein the outer or/and inner wall (3 or/and 4) comprises several, i.e. 2, 3, 4 or more segments.

7. Device according to claim 6, wherein the several segments are realized to be connected to each other via bending means (10) or/and means of rotation (12) or/and elements for the reduction of volume (7) and are, thus, realized to be able to be bent or/and rotated against one another, as well as to be variable in the length of the individual segments or in the length of several connected segments.

8. Device according to claim 1, wherein the attachment means (2, 2a) are realized as means of vacuum or/and means of clamping or/and means of at least temporary adhesion.

9. Device according to claim 1, wherein the attachment means (2, 2a) comprise sensors, e.g. in the form of vacuum sensors or/and force/time/power/torque readers or/and optic sensors for the surveillance of the degree of attachment.

10. Device according to claim 6, wherein the device is assigned a display device for the attachment.

11. Device according to claim 1, wherein the device is realized for the guidance of several instruments within the wall (3a, b or 4).

12. Device according to claim 10, wherein the display device comprises means giving light, sound, scent or tactile signals (e.g. vibrations) as a display means.

13. Medical device or device for the examination of cavities, comprising a device according to claim 1.

14. Use of a device according to claim 1 for the examination or manipulation of walls of cavities.

15. Method for the guidance of instruments for the examination or manipulation of walls of cavities, characterized by the following principal steps:
a) at least partial insertion of the device according to claim 1
b) activation of the—in the desired guidance direction—rearmost attachment means of the device for the attachment of a part of the device to a part of the cavity wall
c) forward movement of the part of the device not attached to the wall (e.g. the inner wall with previous attachment of the outer wall, or e.g. the fore part of the outer wall with previous attachment of the rear part of the wall, or e.g. the instrument with the attachment means arranged on that)
d) attachment of the moved wall or the moved instrument to a cavity wall
e) detachment of the attachment from partial step b) and, thus, the corresponding part of the device
f) subsequent movement of the previously not moved parts of the device
g) repetition of steps a-f.
